(19) United States
(12) Patent Application Publication
(10) Pub. No.: US 2017/0136036 A1
(43) Pub. Date: May 18, 2017

(54) Method for providing regular contraception

(71) Applicant: LABORATOIRE HRA-PHARMA, Paris (FR)

(72) Inventors: Delphine LEVY, Bagnolet (FR); Erin GAINER, Chardonne (CH); Andre ULMANN, Paris (FR)

(21) Appl. No.: 15/322,361

(22) PCT Filed: Jul. 2, 2015
(86) PCT No.: PCT/EP2015/065079

§ 371 Date: Dec. 27, 2016

(30) Foreign Application Priority Data
Jul. 3, 2014 (EP) 14306086.1

(51) Int. Cl. A61K 31/573; A61K 9/00

(52) U.S. Cl. CPC A61K 31/573; A61K 9/0053

(57) ABSTRACT

The invention relates to a method for providing regular contraception to a woman. Said method comprises administering a daily contraceptive amount of ulipristal acetate or a metabolite thereof over a period of at least 21 consecutive days.

### FIELD OF THE INVENTION

The present invention relates to a method for providing regular contraception to a woman, and to contraceptive kits.

### TECHNOLOGICAL BACKGROUND

The combined oral contraceptive pills, also called birth-control pills, are currently used by more than 100 million women worldwide and by almost 12 million women in the United-States. These pills comprise a combination of two contraceptive agents, namely an estrogen and a progestogen. However, combined contraceptive pills display drawbacks in terms of side-effects. These pills may increase the risk of venous thromboembolism, hypertension and cardiovascular diseases, in particular in overweight women. These side-effects are mostly induced by the estrogen compound. Over the past decades, several progestogen-only pills (also called mini-pills or POP) were also developed. Despite their better safety, the use of progestogen-only pills remains marginal. In the United States, the progestogen-only pills are mainly indicated for breast-feeding women and women who cannot tolerate estrogen. Indeed, the progestogen-only pills display a lower contraceptive reliability than combined oral pills. They must be taken at the same time, every day, without any pill-free or placebo period. Moreover, the progestogen-only pills generally alter the bleeding patterns as compared to natural menstrual cycles, by inducing irregular bleedings and spotting, which is very disturbing for women. Progestogen-only pill may also display a poor breast tolerance and induce breast pain (mastodynia).

There is still a need for alternative contraceptive methods, in particular estrogen-free contraceptive methods.

### SUMMARY OF THE INVENTION

The invention relates to a method for providing contraception to a woman, comprising daily administering said woman with a contraceptive agent selected from 17α-acetoxy-11β-[4-N, N-dimethylamino-phenyl]-19-norpregna-4, 9-diene-3, 20-dione (ulipristal acetate) and a metabolite thereof, over a period of 21 to 28 consecutive days. Preferably, the administration is performed by oral route. The daily amount of the contraceptive agent is typically from about 2 mg to 12 mg, preferably from 4 mg to 10 mg.

In some embodiments, the method of the invention comprises:
a first phase wherein the contraceptive agent is daily administered to the woman over a period of 21 to 27 consecutive days, followed by
a second phase selected from:
   a phase wherein no contraceptive agent is administered over a period of 1 to 7 consecutive days; or
   a phase wherein a low daily dosage of a contraceptive is administered over a period of 1 to 7 consecutive days.

The first phase of said method may last 24 consecutive days and the second phase may last 4 consecutive days.

In some embodiments, the daily dosage of ulipristal acetate is from 0.01 mg to 12 mg, preferably from 2 mg to 12 mg, e.g. 3 to 10 mg in the first phase.

For instance, the daily dosage of ulipristal acetate may be 4.0, 5.0, 8.0 or 10.0 mg in the first phase.

In some embodiments, no contraceptive agent is administered during the second phase. Instead, a daily placebo unit may be administered to the woman during the second phase of the method.

In some other embodiments, a contraceptive is administered in the second phase, at a dosage which is at least 1.5-fold, preferably at least 5-fold lower than the dosage administered in the first phase. In particular, the dosage of the contraceptive administered during the second phase may be a non-contraceptive dosage.

The method of the invention is preferably an estrogen-free contraceptive method. Ulipristal acetate or said metabolite thereof is typically the sole contraceptive agent administered to the woman.

For instance, the same contraceptive may be administered during the first and the second phases. Preferably said contraceptive is ulipristal acetate.

The method of the invention may be repeated over several consecutive months, even over several consecutive years.

In a particular aspect, the invention relates to a method for providing contraception to a woman, which comprises:
a first phase wherein ulipristal acetate is daily administered to the woman by oral route over a period of 24 consecutive days, followed by
a second phase wherein no contraceptive amount of the contraceptive agent is administered over a period of 4 days.

A further object of the invention is a contraceptive kit suitable for implementing a method for providing regular contraception as defined herein. Said contraceptive kit comprises one or several packaging units, each packaging unit comprising from 21 to 28 daily dosage units of ulipristal acetate or a metabolite thereof. In some embodiments, each packaging unit comprises from 21 to 27 daily dosage units and optionally from 1 to 7 daily placebo units. For instance, each packaging unit may comprise 24 daily dosage units of ulipristal acetate or a metabolite thereof and 4 daily placebo units. The packaging units may be blister packs.

### DETAILED DESCRIPTION OF THE INVENTION

Ulipristal acetate (also called herein UPA) is a selective progesterone receptor modulator. Ulipristal acetate has been approved by the European Medecines Agency as an emergency contraceptive (EllaOne®) and for the treatment of uterine fibroids (Esmya®). As an emergency contraceptive, ulipristal acetate is to be administered in a single dosage of 30 mg within 120 h after an unprotected intercourse. Repeated uses of ulipristal acetate within the same menstrual cycle are not recommended. For the treatment of uterine fibroids, ulipristal acetate is used for a maximum of 3 months to reduce the size of fibroids, to stop or reduce bleeding and to improve hematocrit level, before myomectomy. To the knowledge of the Inventors, no regular contraceptive method based on the administration of a SPRM, including ulipristal acetate, has been developed and marketed until now. While seeking a contraceptive alternative to combined oral pills and progestogen-only pills disclosed in the prior art, the Inventors studied whether ulipristal acetate could be used as a regular contraceptive. Based on these studies, the Inventors conceived a new contraceptive method based on the administration of a low daily amount of ulipristal acetate over a period of at least 21 days. The co-administration of ulipristal acetate with an estrogen is not required to achieve contraception. Thus, the method of the invention is suitable for women for whom the administration of estrogens is not recommended, for instance, women predisposed to cardiovascular diseases especially deep venous thrombosis, history of breast cancer, or high weight/obese women. The method of the invention is expected to be safer than standard oral contraceptives in terms of venous thromboembolism and cardiovascular risk, while displaying a contraceptive reliability higher than that of progestogen-only pill.

Methods for Contraception According to the Invention

In a first aspect, the present invention relates to a method for providing regular contraception to a woman, comprising administering said woman, with a daily amount of a contraceptive agent over a period of at least 21 consecutive days.

As used herein, a regular contraceptive method refers to a method able to prevent pregnancy in a woman of child-bearing age over at least one menstrual cycle. A woman of child-bearing age refers to a woman from puberty to menopause. In the context of the instant invention, the contraceptive agent refers to ulipristal acetate or a metabolite thereof. Ulipristal acetate, formerly known as CDB-2914, is 17α-acetoxy-11β-[4-N, N-dimethylamino-phenyl]-19-norpregna-4, 9-diene-3, 20-dione, represented by formula I:

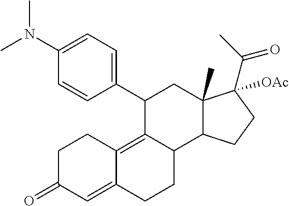 [SMILES: CC(=O)O[C@]1(C(C)=O)CCC2C3CCC4=CC(=O)CCC4=C3C(c3ccc(N(C)C)cc3)C[C@@]21C]

This compound, and methods for its preparation, are described in U.S. Pat. Nos. 4,954,490, 5,073,548, and 5,929,262, and international patent applications WO2004/065405 and WO2004/078709. Metabolites of CDB-2914, include those described in Attardi et al, Journal of Steroid Biochemistry & Molecular Biology, 2004, 88: 277-288, e.g. monodemethylated CDB-2914 (CDB-3877); didemethylated CDB-2914 (CDB-3963); 17alpha-hydroxy CDB-2914 (CDB-3236); aromatic A-ring derivative of CDB-2914 (CDB-4183).

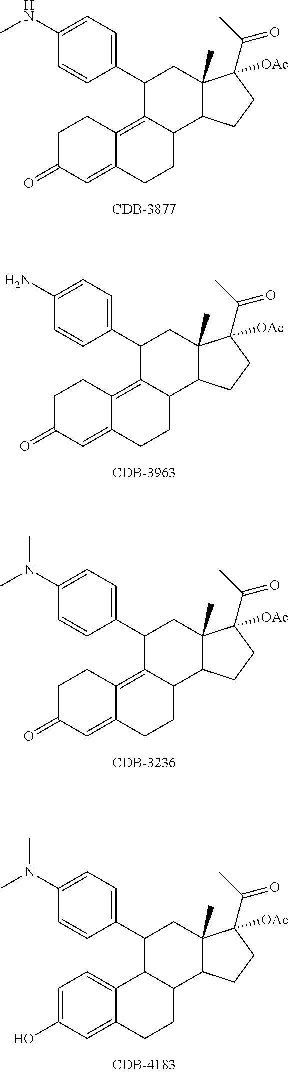 [SMILES: CNc1ccc(C2C[C@@]3(C)C(CC[C@]3(OC(C)=O)C(C)=O)C3CCC4=CC(=O)CCC4=C23)cc1 ; CC(=O)O[C@]1(C(C)=O)CCC2C3CCC4=CC(=O)CCC4=C3C(c3ccc(N)cc3)C[C@@]21C ; CC(=O)O[C@]1(C(C)=O)CCC2C3CCC4=CC(=O)CCC4=C3C(c3ccc(N(C)C)cc3)C[C@@]21C ; CC(=O)O[C@]1(C(C)=O)CCC2C3CCc4cc(O)ccc4C3C(c3ccc(N(C)C)cc3)C[C@@]21C]

In a preferred embodiment, the contraceptive agent is selected from ulipristal acetate, 17α-acetoxy-11β-[4-N-methylamino-phenyl]-19-norpregna-4,9-diene-3,20-dione (CDB-3877) and 17α-acetoxy-11β-[4-aminophenyl]-19-norpregna-4, 9-diene-3, 20-dione (CDB-3963). In a more preferred embodiment, the contraceptive agent is ulipristal acetate.

In some preferred embodiments, ulipristal acetate or said metabolite thereof is not co-administered with an estrogen agent. In other words, the method for providing regular contraception according to the invention is preferably an estrogen-free contraceptive method. As used herein, an estrogen agent refers to a compound able to bind and activate estrogen receptor such as ethinylestradiol, mestranol or phytoestrogens such as 8-prenylnaringenin. In some other embodiments, ulipristal acetate or a metabolite thereof is the sole selective progesterone receptor modulator (SPRM) which is administered when implementing the contraceptive method of the invention. SPRM encompasses, without being limited to, mifepristone, telapristone, asoprisnil, onapristone, Org 33628, Org 31710 and the like.

In some further embodiments, ulipristal acetate or said metabolite thereof is the sole contraceptive agent administered when carrying out the method of the invention. In particular, the method of the invention may be an estrogen-free and progestogen-free contraceptive method. Progestogens encompass pure agonists of the progesterone receptor such as progesterone and derivatives thereof, norethindrone, norethindrone acetate, ethynodiol diacetate, dydrogesterone, medroxy-progesterone acetate, norethynodrel, allylestrenol, lynoestrenol, medrogestone, norgestrienone, dimethiderome, ethisterone, cyproterone acetate, levonorgestrel, gestodene, desogestrel, norgestimate, nestorone, dienogest and drospirenone.

Ulipristal acetate or said metabolite thereof may be administered by any appropriate route, including oral, buccal, sublingual, parenteral, transdermal, vaginal, rectal, subcutaneous, intra-peritoneal etc.

In a preferred embodiment, the contraceptive agent is administered by oral route.

Any appropriate oral dosage forms can be used for implementing the method of the invention. Examples are provided hereunder in the section entitled “Contraceptive compositions for implementing the method of the invention”.

In some embodiments, the method of the invention comprises the daily administration of ulipristal acetate or a metabolite thereof by oral route over a period of at least 21 consecutive days. In a preferred embodiment, ulipristal acetate or the metabolite thereof is administered once a day, over a period of at least 21 consecutive days.

The daily dosage of ulipristal acetate or a metabolite thereof is selected so as to prevent pregnancy during the duration of the method of the invention (typically 28 days). The prevention of pregnancy may be obtained by various biological effects such as the thickening of cervical mucus (which reduces the sperm viability and penetration) and the inhibition of ovulation. In a preferred embodiment, the daily dosage of ulipristal acetate or a metabolite thereof is selected so as to inhibit ovulation.

The daily dosage of ulipristal acetate typically ranges from about 0.01 mg to about 12 mg. The daily dosage may be adjusted depending on individual factors such as the age, the body weight, more precisely the body mass index (BMI), the general health and the diet of the woman.

It goes without saying that the daily dose is also function of the route of administration. For oral administration, the daily dosage of ulipristal acetate may be from 2 mg to 12 mg. In a preferred embodiment, the daily dosage of ulipristal acetate or a metabolite thereof ranges from about 3 mg to about 10 mg, such as 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, and 10.0 mg. For instance, ulipristal acetate or a metabolite is administered orally in a daily amount from 3.0 mg to 10.0 mg, for instance from 3.5 to 8.5 mg such as 4.0 mg or 8.0 mg.

For vaginal route, the daily dosage of ulipristal acetate may be from 0.01 mg to 5 mg.

The contraceptive method of the invention is implemented at least over a period corresponding to the length of a menstrual cycle. The length of the menstrual cycle may vary among women, and typically ranges from 24 days to 35 days. The average length is about 28 days. The first day of the menstrual cycle correlates with the first day of the menses. The contraceptive method of the invention is thus generally performed for a period of time corresponding to the average length of a menstrual cycle i.e. 28 days and may be repeated during several consecutive months. In some preferred embodiments, the contraceptive method of the invention is repeated over at least three consecutive months, preferably over at least six consecutive months, for instance over at least one year. When carried out for the first time, the contraceptive method of the invention may begin within the first five days of the menstrual cycle. The contraceptive method preferably begins on the first day of the menses of the woman.

In some embodiments, the method for providing regular contraception to a woman comprises the daily administration of ulipristal acetate or a metabolite thereof over a period of 28 consecutive days and can be repeated without any contraceptive-free period. In other words, in such an embodiment, the method of the invention comprises the daily administration of ulipristal acetate or a metabolite thereof over a period of 28 consecutive days without any contraceptive-free period, i.e. a period in which no contraceptive is administered.

However, it is expected that a contraceptive-free period or a period wherein a lower dosage of contraceptive is administered may be beneficial to the woman. Without being bound by any theory, the Inventors expect that such a period, in particular a contraceptive-free period, would improve the safety and the tolerability of the contraceptive method. For instance, such a period may prevent any non-physiological change of the endometrium such as benign thickening cystic glandular dilation, or vascular architecture changes. Moreover, such a period may also improve the bleeding pattern of the woman by allowing regular bleedings to occur so as to reproduce the menses, without any decrease of the contraceptive efficacy.

In other words, such a method is expected to display a high contraceptive efficacy without the drawbacks (e.g. spotting, irregular bleedings, strict timing of administration . . . ) observed for progestogen-only pills.

Accordingly, in some preferred embodiments, the method for providing a regular contraception according to the invention comprises two consecutive phases:
a first phase wherein the contraceptive agent is daily administered to the woman over a period of 21 to 27 consecutive days, and
a second phase selected from:
   a phase wherein no contraceptive agent is administered over a period of 1 to 7 consecutive days or
   a phase wherein a low daily dosage of a contraceptive is administered over a period of 1 to 7 consecutive days.

Preferably, the total of the first phase and the second phase of the method of the invention is 28 days.

As used herein, a period of 1 to 7 consecutive days include a period of 1 day, of 2 consecutive days, of 3 consecutive days, of 4 consecutive days, of 5 consecutive days, of 6 consecutive days, and of 7 consecutive days.

As used herein a period of 21 to 27 consecutive days include a period of 21 consecutive days, of 22 consecutive days, of 23 consecutive days, of 24 consecutive days, of 25 consecutive days, of 26 consecutive days, and of 27 consecutive days.

The duration of the first phase plus the second phase is preferably 28 days.

In some embodiments, the first phase lasts from 24 to 27 consecutive days and the second phase lasts from 1 to 4 consecutive days with the proviso that the overall duration of the first phase plus the second phase is 28 days.

For instance, the first phase may last 24 days and the second phase may last 4 days.

The daily amount of ulipristal acetate or a metabolite thereof may be the same along all the first phase or may change. In a preferred embodiment, the same amount of ulipristal acetate of a metabolite thereof is administered each day of the first phase. As mentioned above, a daily dosage of 4 mg to 12 mg of the contraceptive agent may be administered during the first phase, by oral route.

In some embodiments, no contraceptive is administered to the woman in the second phase of the contraceptive method. The second phase is thus a contraceptive-free period. In some embodiments, during said second phase, a daily placebo unit is administered to the woman. In some other embodiments, no pill, more precisely no placebo, is administered to the woman during the second phase.

In a preferred embodiment, the method for providing a regular contraception of the invention comprises two consecutive phases:
a first phase wherein ulipristal acetate or a metabolite thereof is daily administered to the woman over a period of 24 to 27 consecutive days, preferably 24 consecutive days, and
a second phase wherein no contraceptive agent is administered over a period of 1 to 4 consecutive days, preferably 4 consecutive days.

As mentioned above, the contraceptive method according to the invention may be repeated during several consecutive months. Accordingly, in a more general aspect, the invention also relates to a method for providing contraception to a woman, said method comprises one or more consecutive cycles, each cycle lasting 28 consecutive days and comprising:
a first phase wherein ulipristal acetate or a metabolite thereof is administered to the woman over a period of 21 to 27 consecutive days, and
a second phase wherein no contraceptive agent is administered over a period of 1 to 7 consecutive days.

In some embodiments said method comprises at least 3 cycles, such as at least 6 cycles and even at least 12 cycles.

It goes without saying that said method may have one or several of the specific features previously described, such as:
the daily dosage of ulipristal acetate or a metabolite thereof in the first phase ranges from 0.01 to 12 mg, preferably from 2 to 12 mg, e.g. 3 to 10 mg, and/our
the administration of ulipristal acetate or a metabolite thereof is oral and/or
the first phase lasts 24 days, and the second phase last 4 days, and/or
daily placebo unit is administered during the second phase.

In some other embodiments, the method for providing a regular contraception according to the invention comprises two consecutive phases:
a first phase wherein the contraceptive agent is administered to the woman over a period of 21 to 27 consecutive days, and
a second phase wherein a low daily dosage amount of a contraceptive agent is administered over a period of 1 to 7 consecutive days.

Preferably, the same contraceptive, e.g. ulipristal acetate, is administered in the first and the second phases. Generally, the low daily dosage administered in the second phase refers to a daily dosage lower than that administered in the first phase.

For instance, the method for providing a regular contraception according to the invention comprises two consecutive phases:
a first phase wherein the contraceptive agent is administered to the woman over a period of 24 to 27 consecutive days, and
a second phase wherein the contraceptive agent is administered over a period of 4 to 7 consecutive days, the contraceptive agent being administered at a dosage lower than that administered in the first phase.

In some embodiments, the daily dosage administered during the second phase may be at least 1,5-fold (i.e. 2, 3, 4, 5, 6, 8, 10, 15 or 20-fold) lower than that administered in the first phase. For instance the daily dosage of ulipristal acetate in the first phase may be from 5 to 10 mg and the daily dosage of ulipristal acetate administered in the second phase may be from 0.1 to 5 mg. For instance, the daily dosage in the first phase may be 5 mg while the daily dosage administered in the second phase may be 0.1 to 2 mg.

In some embodiments, the daily dosage of the contraceptive administered in the second phase is a non-contraceptive dosage.

A non-contraceptive dosage refers to a dosage which is not sufficient to prevent pregnancy over the length of one menstrual cycle, when daily administered to a woman during one menstrual month. By contrast, a daily contraceptive dosage refers to a dosage which is sufficient to prevent pregnancy, preferably by inhibiting ovulation, over the length of one menstrual cycle, when daily administered to a woman during one menstrual month.

In another aspect, the instant invention relates to the use of ulipristal acetate or a metabolite thereof for providing regular contraception to a women, wherein ulipristal acetate or a metabolite thereof is administered to the woman over a period of at least 21 consecutive days, typically from 21 to 28 consecutive days.

In a further aspect, the invention relates to the use of ulipristal acetate or a metabolite thereof in the manufacture of a regular contraceptive, wherein said contraceptive is administered to a woman over a period of at least 21 consecutive days, typically from 21 to 28 consecutive days.

It goes without saying that the uses of the invention may display any of the specific features disclosed herein for the contraceptive methods according to the invention.

Contraceptive Compositions for Implementing the Method of the Invention

As mentioned above, ulipristal acetate or a metabolite thereof may be administered by any convenient route. Any type of pharmaceutical forms containing ulipristal acetate or a metabolite thereof may be used for implementing the contraceptive method of the invention. Such forms encompass suppositories, injectable solutions, syrups, creams, transdermal patches, transdermal spray, capsules, tablets, vaginal tablets, mucoadhesive tablets, powders, suspensions, granules, and the like.

The pharmaceutical form may be also incorporated in a vaginal ring or in an intra-uterine device.

In a preferred embodiment, the pharmaceutical form is an oral form, preferably a solid oral form, such as a coated or uncoated tablet or a capsule.

Ulipristal acetate or a metabolite thereof may be formulated according to standard methods as described in Remington: The Science and Practice of Pharmacy (Lippincott Williams & Wilkins; Twenty first Edition, 2005). Pharmaceutically acceptable excipients that may be used to formulate the contraceptive compositions of the invention are, among others, described in the Handbook of Pharmaceuticals Excipients, American Pharmaceutical Association (Pharmaceutical Press; 6th Revised edition, 2009).

Examples of appropriate excipients include, but are not limited to, fillers, carriers, diluents, binders, anti-caking agents, plasticizers, disintegrants, lubricants, flavors, buffering agents, stabilizers, colorants, dyes, anti-oxidants, anti-adherents, softeners, preservatives, surfactants and glidants.

In some embodiments, the contraceptive composition comprises one or more excipients selected from the group of binders, diluents, disintegrants, glidants and lubricants.

Examples of diluents include, without being limited to, microcrystalline cellulose, starch, modified starch, dibasic calcium phosphate dihydrate, calcium sulfate trihydrate, calcium sulfate dihydrate, calcium carbonate, mono- or disaccharides such as lactose, dextrose, sucrose, mannitol, galactose and sorbitol, xylitol and combinations thereof.

Examples of binders include, without being limited to, starches, e.g., potato starch, wheat starch, corn starch; gums, such as gum tragacanth, acacia gum and gelatin; hydroxypropyl cellulose, hydroxyethyl cellulose, hydroxypropyl methyl cellulose; polyvinyl pyrrolidone, copovidone, polyethylene glycol and combinations thereof.

Examples of lubricants include, without being limited to, fatty acids and derivatives thereof such as calcium stearate, glyceryl monostearate, glyceryle palmitostearate magnesium stearate, zinc stearate, or stearic acid, or polyalkyleneglycols such as PEG.

The glidant may be selected among colloidal silica, dioxide silicon, talc and the like.

Examples of disintegrants encompass, without being limited to, crospovidone, croscarmellose salts such as sodium croscarmellose, starches and derivatives thereof.

Examples of surfactants encompass, without being limited to, simethicone, triethanolamine, les polysorbate and derivatives thereof such as Tween® 20 or Tween® 40, poloxamers, fatty alcohol such as laurylic alcohol, cetylic alcohol and alkylsulfate such as sodium dodecylsulfate (SDS).

The pharmaceutical composition may be obtained merely by mixing ulipristal acetate or a metabolite thereof with one or several excipients and may be shaped by standard methods. For instance, a tablet may be obtained by wet granulation, dry granulation or direct compression.

Ulipristal acetate or a metabolite thereof may be micronized, co-micronized with a suitable excipient, e.g. a surfactant such as SDS, or provided as a solid dispersion.

The contraceptive composition may comprise:
from 0.5% to 70% by weight of ulipristal acetate or a metabolite thereof,
from 0% to 10% by weight of desintegrant,
from 15% to 98% by weight of diluent,
from 0% to 5% by weight of lubricant,
from 0% to 10% by weight of binder,
from 0% to 5% by weight of glidant, and
from 0% to 50% by weight of surfactant, the percentage being expressed as compared to the total weight of the composition.

As mentioned above, ulipristal acetate may be present as micronized ulipristal acetate, as a co-micronized mixture or as a solid dispersion. Preferably, ulipristal acetate is present as micronized ulipristal acetate or as a comicronized mixture with SDS, the weight ratio of ulipristal acetate to SDS ranging from 0.5 to 4, preferably from 0.8 to 1.2.

In some embodiments, the contraceptive composition may comprise:
from 1% to 20% by weight of ulipristal acetate or a metabolite thereof,
from 50% to 95% of a diluent, preferably selected from lactose, mannitol microcrystalline cellulose, and combinations thereof
from 0% to 10% of a binder, preferably selected from povidone, PEG, HPMC, copovidone and combinations thereof,
from 0.5% to 10% of a disintegrant preferably selected from sodium croscarmellose and crospovidone, and
from 0.5% to 5% of a lubricant, preferably magnesium stearate.

In preferred embodiments, the contraceptive composition is in the form of a coated tablet or an uncoated tablet. In some alternate embodiments, the contraceptive composition is in the form of a powder or granules contained within a capsule, such as a gelatin capsule.

Accordingly, a daily dosage unit for implementing the method of the invention may be in the form of a tablet (or pill), or a capsule. As mentioned above, a daily dosage unit may comprise from 4 mg to 12 mg of ulipristal acetate or a metabolite thereof, e.g. 5 mg or 10 mg of ulipristal acetate or metabolite thereof.

In some additional embodiments, the contraceptive composition is an immediate-release one. Accordingly, the contraceptive composition of the invention is characterized by a rapid dissolution rate of ulipristal acetate or said metabolite thereof in vitro. As used herein, an “immediate-release composition” refers to a pharmaceutical composition wherein at least 75% of the active ingredient contained in a dosage unit of said composition is released within 45 minutes when said dosage unit is subjected to an in vitro dissolution assay according to the European Pharmacopeia §2.9.3.

In some embodiments, the pharmaceutical composition is such that at least 80% of ulipristal acetate or metabolite thereof initially contained in a dosage unit of said composition is released within 20 minutes, preferably within 10 minutes, when said dosage unit is subjected to an in vitro dissolution assay according to the European Pharmacopeia §2.9.3.

In some embodiments, the composition of the invention is estrogen-free, this means that said composition is devoid of estrogen.

In some additional embodiments, ulipristal acetate or a metabolite thereof is the sole contraceptive present within the composition.

Contraceptive Kits According to the Invention

In a further aspect, the invention relates to a contraceptive kit suitable for implementing a contraceptive method according to the instant invention. Said contraceptive kit comprises at least 21, typically from 21 to 28, daily dosage units comprising ulipristal acetate or a metabolite thereof. The daily dosage units are preferably solid dosage units for oral administration such as tablets (or pills) as well as capsules. The daily dosage units may be made of a contraceptive composition as described hereabove.

In some embodiments, the contraceptive kits may comprise at least one packaging unit.

At least one packaging unit includes, without being limited to, 1 packaging unit, 2 packaging units, 3 packaging units, 4 packaging units, 5 packaging units and 6 packaging units.

Each packaging unit comprises from 21 to 28 daily active dosage units. Each packaging unit may optionally comprise from 1 to 7 daily dosage units of a pharmaceutically acceptable placebo.

In some embodiments, the contraceptive kit is characterized in that each packaging unit comprises 28 daily dosage units and no daily dosage unit of a pharmaceutically acceptable placebo. Such a contraceptive kit is particularly appropriate to perform the contraceptive method of the invention which consists in administering “continuously” ulipristal acetate or a metabolite thereof without any contraceptive-free period.

In some preferred embodiments, each packaging unit of the kit comprises:
21 to 27 daily dosage units of ulipristal acetate or a metabolite thereof and
optionally 1 to 7 daily placebo units.

Such a contraceptive kit is particularly appropriate to perform the contraceptive method of the invention which comprises:
a first phase wherein the contraceptive agent is administered to the woman over a period of 21 to 27 consecutive days, and
a second phase wherein no contraceptive agent is administered over a period of 1 to 7 consecutive days.

When the packaging unit comprises one or several placebo units, the sum of the daily placebo units and the daily dosage units is preferably 28.

In some preferred embodiments, each packaging unit of the kit comprises 24 daily dosage units comprising ulipristal acetate or a metabolite thereof and, optionally, 4 daily placebo units.

The composition of the placebo units may be similar to that of the daily dosage unit, except that said placebo units are devoid of any active ingredient, including ulipristal acetate or a metabolite thereof.

In other embodiments, each packaging unit of the kit comprises:
21 to 27 first daily dosage units of ulipristal acetate or a metabolite thereof and
1 to 7 second daily dosage units comprising ulipristal acetate or a metabolite thereof in an amount lower than that contained in the first daily dosage units.

For instance, each first daily dosage unit may contain from 5 mg to 10 mg of ulipristal acetate or a metabolite thereof and each second daily dosage unit may contain from 0.1 mg to 5 mg of ulipristal acetate or a metabolite thereof.

The packaging unit as described above may have one of the conventional forms usually used for oral contraceptives. For example, the packaging unit may be a conventional blister pack comprising the appropriate number of dosage units in a sealed blister pack with a cardboard, paperboard, foil or plastic backing and enclosed in a suitable cover. Each blister pack may be conveniently numbered or marked in order to facilitate compliance. The packaging unit may contain daily dosage units in the order in which they have to be taken, i.e. starting with the first of the at least 24 dosage units that contains ulipristal acetate or a metabolite thereof optionally followed by 4 placebo dosage units. The kit of the invention may comprise other appropriate components such as instructions for use.

The following examples are provided by way of illustration only and not by way of limitation.

### Example 1: Tablets Suitable for Implementing the Contraceptive Method of the Invention

The tables hereafter show some tablets which may be prepared by direct compression. The co-micronized mixture of ulipristal acetate with SDS may be obtained as described in PCT/FR2013/052670:

TABLE 1

Daily dosage units

| | mg/tablet | % by weight |
|---|---|---|
| Comicronized UPA/SDS 3/2 | 10.00 | 6.7 |
| | (6 mg of UPA) | |
| Microcrystalline cellulose | 92.45 | 61.6 |
| Mannitol | 43.50 | 29 |
| Sodium croscarmellose | 2.55 | 1.7 |
| Magnesium stearate | 1.50 | 1.00 |
| Total | 150.00 | 100 |

TABLE 2

Daily dosage units

| | mg/tablet | % by weight |
|---|---|---|
| Comicronized UPA/SDS 1/1 | 8.00 | 6.7 |
| | (4 mg of UPA) | |
| Lactose | 28.80 | 45 |
| Mannitol | 20.48 | 32 |
| Sodium croscarmellose | 6.40 | 10 |
| Magnesium stearate | 0.32 | 0.5 |
| Total | 64.00 | 100 |

TABLE 3

Daily dosage units

| | mg/tablet | % by weight |
|---|---|---|
| Comicronized UPA/SDS 1/1 | 10.00 | 6.67 |
| | (5 mg of UPA) | |
| Microcrystalline cellulose | 88.25 | 58.83 |
| Mannitol | 43.50 | 29.00 |
| Crospovidone | 7.5 | 5.00 |
| Magnesium stearate | 0.75 | 0.50 |
| Total | 150.00 | 100 |

The tablets may be also obtained by wet granulation and may have the following composition:

TABLE 4

Daily dosage units

| | mg/tablet | % by weight |
|---|---|---|
| Micronized ulipristal acetate | 10.00 | 10.00 |
| Lactose Monohydrate | 79.00 | 79.00 |
| Povidone | 5.00 | 5.00 |
| Croscarmellose sodium | 5.00 | 5.00 |
| Magnesium stearate | 1.00 | 1.00 |
| Total | 100.00 | 100 |

Any other homothetic tablets of the above compositions can be prepared.

### Example 2: A Contraceptive Kit of the Invention

The contraceptive kit comprises three blister packs. Each blister pack comprises 28 blister pockets. The blister pockets are arranged to house a sequence of 24 tablets each providing a daily dose of 5 mg of ulipristal acetate followed by 4 placebo tablets. The contraceptive kit further contains instructions for use dedicated to the female patient.

### Example 3: A Phase IIb Randomized, Double Blind, Comparative Study to Assess the Efficacy, Safety, Tolerability and Inhibition of Ovulation of Two Continuous Regimens of Oral Daily 5 mg or 10 mg of Ulipristal Acetate (UPA), Versus a Dose of 5.0 mg UPA for 24/4 Days

The primary objective of this randomized, double blind comparative study is to compare the pharmacodynamic effects of 2 continuous dose regimens of ulipristal acetate 5.0 and 10.0 mg-only oral contraception, versus a 24/4 day regimen of UPA 5.0 mg. Secondarily the tolerability and effects of the three different doses is compared for bleeding, follicle growth, endometrial safety and subject satisfaction. The studies comprise a total of 84 days of treatment followed by a two and a half months post treatment follow up of endometrial safety.

The total duration of the study for each participant is expected to be approximately 6.5 months: One month for screening to meet enrolment criteria, three months of treatment with the study product, and a recovery period lasting until the week following the second spontaneous (no hormonal contraceptive) menstrual bleeding episode (expected to take 2.5 calendar months). After enrolment, the subjects are seen twice weekly for a period up to 17 weeks, followed by a final visit to occur 4-6 weeks later depending on an individual subject's normal menstrual cycle length.

The study treatment boxes contain 4 identical blisters containing 28 tablets numbered sequentially 1 to 28 days. Each study treatment box contains one of the following dosing regimens:
4 blisters of 28 tablets of ulipristal acetate 5 mg
4 blisters of 28 tablets of ulipristal acetate 10 mg
4 blisters of 24 tablets of ulipristal acetate 5 mg and 4 placebo pills

Each participant is provided with one blister pack with one backup pack the first month. A single blister pack is then provided for months 2 and 3 if the subject has a full back-up pack remaining. If the back-up pack is not full, the original back-up pack should be returned and a new back-up should be provided.

In order to compare the three methods, each enrolled patient is subjected to the following analysis:
The progesterone levels in serum are determined twice weekly.
The ovarian follicular activity is assessed by transvaginal ultrasounds twice a week.
For the assessment of the bleeding profile, the total number of bleedings and spotting days per 28 days are reported as well as menses duration and intensity, bleeding-related adverse events, hematocrit levels, subject's acceptability of treatment.
The enrolled patient completes a treatment acceptability questionnaire.
The endometrial safety is assessed by performing endometrial biopsies and by determining endometrial thickness and histology by transvaginal ultrasounds.

1. A method for providing contraception to a woman, comprising daily administering said woman, with a contraceptive agent selected from 17α-acetoxy-11β-[4-N,N-dimethylamino-phenyl]-19-norpregna-4, 9-diene-3, 20-dione (ulipristal acetate) and a metabolite thereof, over a period of 21 to 28 consecutive days.

2. The method of claim 1 wherein the contraceptive agent is administered orally.

3. The method of claim 1 which comprises:
a first phase wherein the contraceptive agent is daily administered to the woman over a period 21 to 27 consecutive days, followed by:
a second phase selected from:
a phase wherein no contraceptive agent is administered over a period of 1 to 7 consecutive days; and
a phase wherein a low dosage of a contraceptive is daily administered over a period of 1 to 7 consecutive days.

4. The method of claim 3 wherein no contraceptive agent is administered during the second phase.

5. The method of claim 3 wherein a daily placebo form is administered during the second phase.

6. The method of claim 3 wherein the first phase lasts 24 consecutive days and the second phase lasts 4 consecutive days.

7. The method of claim 1 wherein the contraceptive agent is ulipristal acetate.

8. The method of claim 3, wherein the daily amount of the contraceptive agent administered in the first phase is from about 0.01 mg to 12 mg.

9. The method of claim 1, said method being an estrogen-free contraceptive method.

10. The method of claim 3, wherein the dosage of the contraceptive administered in the second phase is at least 1,5-fold lower than the dosage administered in the first phase.

11. The method of claim 10, wherein the dosage of the contraceptive administered during the second phase is a non-contraceptive dosage.

12. The method of claim 1, wherein ulipristal acetate or a metabolite thereof is the sole contraceptive agent administered to the woman.

13. The method of claim 1, which is repeated over several consecutive months.

14. A contraceptive kit suitable for implementing a method for providing regular contraception of claim 1, which comprises one or several packaging units, each packaging unit comprising from 21 to 28 daily dosage units comprising ulipristal acetate or a metabolic thereof.

15. The contraceptive kit of claim 14, wherein each packaging unit comprises from 21 to 27 daily dosage units and optionally from 1 to 7 daily placebo units.
